(19) United States
(12) Patent Application Publication
(10) Pub. No.: US 2008/0050434 A1
(43) Pub. Date: Feb. 28, 2008

(54) Novel Composition for Topical Delivery

(76) Inventors: Rajesh Jain, New Delhi (IN); Kour Chand, New Delhi (IN); Sukhjeet Singh, New Delhi (IN); Vaibhav Sihorkar, New Delhi (IN)

Correspondence Address: LADAS & PARRY, 26 WEST 61ST STREET, NEW YORK, NY, 10023, US

(21) Appl. No.: 10/593,136

(22) PCT Filed: Mar. 17, 2005
(86) PCT No.: PCT/IN05/00086

§ 371 Date: Aug. 30, 2007

(30) Foreign Application Priority Data
Mar. 18, 2004 (IN) 501/DEL/2004

(51) Int. Cl. A61K 9/00; A61K 31/04; A61K 31/56; A61K 38/13; A61P 17/00; A61P 37/00; A61P 5/00; A61P 17/06; A61K 9/10; A61K 47/10; A61K 47/14

(52) U.S. Cl. 424/485; 424/484; 424/486; 514/11; 514/179; 514/741; 514/772; 514/785

(57) ABSTRACT

Pharmaceutical composition for topical administration comprising of at least one active ingredient, its salts, esters, hydrates or derivatives; a gelator system consisting of a fiend of surfactants, a solvent system comprising at least one oily component; an aqueous phase; optionally containing one or more stabilizing agent; and other pharmaceutically acceptable excipients; and process for preparing such compositions are provided. Also provided is a method for the management/treatment of fungal, bacterial or microbial infections, inflammations, autoimmune conditions, or hormonal disorders which comprises administering a pharmaceutically effective amount of such pharmaceutical composition to a subject in need of such treatment. The compositions of the present invention are non-greasy and easily water washable, and provides an enhanced localization of hydrophobic and/or amphiphilic active ingredients on the skin.

### FIELD OF THE INVENTION

The present invention relates to topical pharmaceutical compositions, process of preparation of such compositions, and method for the management of microbial and/or fungal infections of the skin layers, inflammations, autoimmune conditions, or hormonal disorders using such compositions. Preferably,.the present invention relates to topical compositions comprising of active ingredient(s) either alone or in combination, that are highly effective in the management of microbial and/or fungal infections of the upper skin layers, particularly epidermis and dermis, autoimmune conditions, or hormonal disorders.

### BACKGROUND OF THE INVENTION

Several topical formulations, especially comprising antifungal, antibacterial or antimicrobial drugs, immunomodulators, or steroids exist in literature but most of them suffer from disadvantages such as instability, poor retention on the skin surface, lack of aesthetic appeal, and difficulty in removal from the skin surface.

Terbinafine hydrochloride is a synthetic allylamine derivative useful as topical antifungal agent. Terbinafine hydrochloride exerts its antifungal effect by inhibiting squalene epoxidase, a key enzyme in sterol biosynthesis in fungi. This action results in a deficiency in ergosterol and a corresponding accumulation of sterol within the fungal cell. Terbinafine has been disclosed in U.S. Pat. No. 4,755,538, which reports a number of methods for the preparation thereof. Several articles have been published emphasizing the pharmaceutical properties of Terbinafine; see Petranyl, G. et al; Science, 1984, 24, 1239; Stutz. A. et al, J. Med. Chem., 1984, 27, 1539.

Topical formulations comprising immunosuppressant drugs such as cyclosporine, tacrolimus, etc. and steroids such as testosterone, etc. which are highly absorbed possess an acceptable aesthetic appeal, and are patient compliant in terms of ease of use and removal from the skin surface, have been difficult to develop, especially due to the large size of the drug molecule or por absorption through the skin. Tacrolimus is macrolide immunosuppressant produced by Streptomyces species. Cyclosporine is a cyclic polypeptide immunosuppressant agent consisting of 11 amino acids. It is produced as a metabolite by the fungus species Beauveria nlyea. No topical composition comprising cyclosporine is available in the market.

U.S. Pat. No. 6,383,471 describes compositions and methods for improved delivery of ionizable hydrophobic therapeutic agents. However these compositions do not require a combination of surfactants as an essential feature of the invention. Also there is no indication of gelation i.e. a formation of especially a structured gel of oily components using mixture of surfactants for the topical delivery of drugs.

U.S. Pat. Nos. 6,451,339, 6,294,192 and 6,309,663 disclose pharmaceutical formulations for administration of hydrophobic lipid-regulating agent, comprising a therapeutically effective amount of the lipid-regulating agent and a carrier, where the carrier is formed from a combination of a hydrophilic surfactant and a hydrophobic surfactant. These compositions use a blend of surfactants; the said compositions upon dilution with aqueous solvent form a clear, aqueous dispersion of the surfactants containing the therapeutic agent. However these compositions neither relate to solvent gelling properties using blends of surfactants nor are they meant for topical use. U.S. Pat. No. 6,455,592 discloses use of penetration agents in dermatological compositions for the treatment of onychomycosis, and corresponding compositions with a pharmaceutically effective amount of Terbinafine hydrochloride, solvent medium comprising water, and at least one straight- or branched-chain C₂-C₈ alkanol and a hydrophilic penetration agent. U.S. Pat. No. 6,309,663 claims triglyceride-free pharmaceutical compositions for enhanced absorption of a hydrophilic therapeutic agent comprising hydrophilic and hydrophobic surfactants. U.S. Pat. No. 6,761,903 describes a pharmaceutical composition comprising a carrier comprising a triglyceride and at least two surfactants, at least one of the surfactants being hydrophilic; and a therapeutically effective amount of a polysaccharide drug, wherein the triglyceride and surfactants are present in amounts such that upon mixing with an aqueous medium in an aqueous medium to carrier ratio of about 100:1 by weight, the carrier forms a clear aqueous dispersion having an absorbance of less than about 0.3 at 400 nm. However, none of the said patents describe compositions which comprise a gelator system consisting of a blend of surfactants, a solvent system comprising at least one oily component; an aqueous phase; optionally containing one or more stabilizing agent; and other pharmaceutically acceptable excipients; wherein the blend of surfactants acts as gelators of the oily component present in the solvent system and lead to the formation of a highly structured gelled composition which provides a uniform and localized release on the skin of the active ingredient.

U.S. Pat. No. 5,446,070 discloses compositions and methods for topical administration of pharmaceutically active agents. However, this is a bio-adhesive composition for topical application and does not essentially contain lipophilic solvents and/or surfactants. U.S. Pat. No. 5,593,680 discloses a cosmetic or dermopharmaceutical compositions in the form of aqueous gels modified by the addition of expanded microspheres.

U.S. Pat. Nos. 5,660,839 and 5,939,083 disclose a nongreasy, nonsticky composition comprising at least one fatty substance and an amount of deformable hollow particulate effective to avoid the greasy and/or sticky feel otherwise attributable to said at least one fatty substance, said deformable hollow particulates comprising a copolymer of vinylidene chloride, acrylonitrile and a (meth) acrylic comonomer. U.S. Pat. No. 5,665,386 discloses use of essential oils to increase bioavailability of oral pharmaceutical compounds but does not disclose usage of a specific blend of surfactants to cause gelation of such oils. U.S. Pat. Nos. 5,681,849 and 5,856,355 disclose topical pharmaceutical compositions comprising Terbinafine in free base form or in acid addition salt form, water, a lower alkanol, and a water-soluble or water miscible nonionic surfactant, wherein no anionic surfactant is present and wherein said composition is an emulsion gel or lotion, further comprising an oil phase and a thickener. However, this invention does not pertain to the use of surfactant blends for the gelation of the solvents as a carrier for hydrophobic drugs.

U.S. Pat. No. 5,385,907 describes an ointment consisting essentially of a tricyclic compound such as tacrolimus, solubilizing and/or absorption-promoting agent selected from the group consisting of a lower alkanediol, a lower alkylene carbonate, an alkane dicarboxylic ester, a higher alkane carboxylic glycerin ester, a higher alkene carboxylic glycerin ester, a higher alkane carboxylic alkyl ester, a higher unsaturated alcohol and an azacycloalkane, and an ointment base selected from the group consisting of oil and fat bases. However, such preparations are oily and adhere to the skin, and are not easily removable upon washing with water.

U.S. Pat. No. 6,022,852 discloses pharmaceutical preparation comprising cyclosporin A, tocopherol polyethylene glycol 1000 succinate, and optionally an emulsifier, with the exception of vegetable oil or fat. U.S. Pat. No. 6,113,921 pertains to pharmaceutical composition for topical or transdermal enhanced effect, which comprises droplets in the sub-micron size range of a water-insoluble drug in an aqueous dispersion system, wherein the droplets consist essentially of about 0.5 to 30% of a first component of an oily liquid comprising the drug, about 0.1 to 10% of a second component of an emulsifier and about 0.05 to 5% of a third component of a non-ionic surfactant, wherein the second and third components are different. U.S. Pat. No. 5,891,846 claims a cyclosporin-containing oil-in-water type emulsion composition comprising cyclosporin, a polyalkyl ester of polycarboxylic acid in the form of a liquid at ambient temperature, at least one oil component, a surfactant and crotamiton. U.S. Pat. No. 5,807,820 describes a topical pharmaceutical composition for dermal administration comprising cyclosporin, a C₁₂₋₂₄ mono- or poly-unsaturated fatty alcohol, and dermally acceptable topical carriers or diluents. U.S. Pat. No. 5,504,068 describes a topical preparation comprising cyclosporin; an organic solvent; and a skin penetration enhancer, said skin penetration enhancer being at least one member selected from the group consisting of alkanolamines and monovalent alcohol esters of myristic acid, adipic acid and sebacic acid.

None of the literature available in the art discloses compositions that comprise of therapeutic agent(s) and a blend of surfactants to produce gelation of solvent component(s) containing the therapeutic agent(s) as essential ingredients, which would lead to highly effective and localized topical preparations for extended duration of activity. Hence, there still exists an unmet need to develop highly effective topical compositions for the management of the anti-microbial, anti-fungal infections, autoimmune conditions, or hormonal disorders which can produce the desired effects for extended periods of time with minimal systemic absorption thus avoiding the undue toxicity of drugs.

SUMMARY OF THE INVENTION

It is an objective of the present invention to provide pharmaceutical composition for topical administration providing an enhanced localization of active ingredient comprising of at least one active ingredient, its salts, esters, hydrates or derivatives; a gelator system consisting of a blend of surfactants, a solvent system comprising at least one oily component; an aqueous phase comprising one or more stabilizing agent; and optionally other pharmaceutically acceptable excipients; wherein the blend of surfactants act as gelators of the oily component present in the solvent system forming a three dimensional network which immobilize the solvent system characterized such that the surfactant gelled oily phase can accommodate the aqueous phase without changing the lipid microenvironment and gel architecture of the composition.

It is also an objective of the present invention to provide process for the preparation of a pharmaceutical composition for topical administration providing an enhanced localization of active ingredient comprising of at least one active ingredient, its salts, esters, hydrates or derivatives; a gelator system consisting of a blend of surfactants, a solvent system comprising at least one oily component; an aqueous phase comprising one or more stabilizing agent; and optionally other pharmaceutically acceptable excipients; wherein the blend of surfactants act as gelators of the oily component present in the solvent system forming a three dimensional network which immobilize the solvent system characterized such that the surfactant gelled oily phase can accommodate the aqueous phase without changing the lipid microenvironment and gel architecture of the composition, which comprises of the following steps:
i. preparation of the oily phase comprising gelator system,
ii. incorporating the active ingredient into the oily phase,
iii. preparation of the aqueous phase comprising stabilizer,
iv. mixing both the oily phase and the aqueous phase with continuous stirring to obtain the desired composition.
   It a further objective of the present invention to provide a method for the treatment of fungal, bacterial or microbial infections, inflammations, autoimmune conditions, or hormonal disorders comprising administering a pharmaceutically effective amount of such pharmaceutical composition to a subject in need of such treatment.

The compositions of the present invention provides an enhanced localization of hydrophobic and/or amphiphilic active ingredients for the management of microbial and/or fungal infections of the skin, or for the treatment of autoimmune or hormonal disorders.

It is still another objective of the present invention to provide essentially non-greasy and easily water washable pharmaceutical compositions meant for topical administration.

### DETAILED DESCRIPTION OF THE INVENTION

The present invention provides pharmaceutical composition for topical administration providing an enhanced localization of active ingredient comprising of at least one active ingredient, its salts, esters, hydrates or derivatives; a gelator system consisting of a blend of surfactants, a solvent system comprising at least one oily component; an aqueous phase comprising one or more stabilizing agent; and optionally other pharmaceutically acceptable excipients.

The blend of surfactants present in the pharmaceutical compositions of the present invention acts as gelators of the oily component present in the solvent system forming a three dimensional network which immobilize the solvent system characterized such that the surfactant gelled oily phase can accommodate the aqueous phase without changing the lipid microenvironment and gel architecture of the composition.

The pharmaceutical compositions of the present invention are preferably a gelled topical system with a rich lipid microenvironment, but easily water washable. In an essential embodiment, the present invention overcomes the problems associated with drug localization in the upper skin layers by providing unique gelator-based lipidic microenvironment. The term ‘gelation’ used herein refers to the gelling of the oily component by the blend of surfactants used in the composition of the present invention leading to the formation of a highly structured system.

The present invention provides pharmaceutical compositions comprising a hydrophobic or amphiphilic active ingredient, selected from but not limited to a group comprising antifungals such as terbinafine, butenafine, griseofulvin, and the like; antibacterials such as sertaconazole, minocycline, and the like; immunomodulators such as cyclosporine, tacrolimus, and the like; steroids such as testosterone, hydrocortisone, and the like; analgesics, anti-inflammatory agents such as nimesulide, diclofenac, ibuprofen, naproxen, and the like; keratinizing agents such as salicylic acid; antimicrobials, skin nourishing or sensitizing agents, anti-psoriatic and anti-eczema drugs, used either alone or in combination thereof. In a preferred embodiment of the present invention, the active ingredient is terbinafine, tacrolimus, cyclosporine, testosterone, hydrocortisone, or salts, esters, hydrates or derivatives thereof.

In an embodiment, the pharmaceutical composition of the present invention comprises a gelator system consisting of a blend of surfactants comprise of at least two surfactants wherein at least one is a hydrophilic surfactant having an HLB value greater than or equal to about 10; and a lipophilic surfactant having an HLB value less than about 10. The lipophilic surfactant component is present in an amount sufficient to achieve the required concentration ratio of the blend of surfactants to bring about the gelation of one or more oily components present in the solvent system.

In another embodiment, the gelator system is present in an amount from about 5% to about 50% by weight of the total weight of composition.

The hydrophilic surfactant of the gelator system of the present invention is selected from but not limited to the group comprising of polyoxyethylene alkyl ethers; polyoxyethylene sorbitan fatty acid esters known as Polysorbates; polyoxyethylene alkyl phenols; polyethylene glycol fatty acid esters; polyglycerol fatty acid esters; polyoxyethylene glycerides; polyoxyethylene sterols; polyoxyethylene vegetable oils; polyoxyethylene hydrogenated vegetable oils; propylene glycol alginate; lecithins and hydrogenated lecithins; lysolecithin and hydrogenated lysolecithins; lysophospholipids and derivatives thereof; phospholipids and derivatives thereof; salts of fatty acids; lauryl macrogolglycerides, or mixtures thereof.

The lipophilic surfactant of the present invention is selected from but not limited to the group comprising of fatty acids; sorbitan fatty acid esters; acetylated glycerol fatty acid esters; lower alcohol fatty acids esters; trans-esterification products of fatty acids, glycerides, vegetable oils, hydrogenated vegetable oils, triglycerides and polyalkylene polyols; sterols and sterol derivatives; pentaerythritol fatty acid esters and polyalkylene glycol ethers; monoglycerides and acetylated, e.g. mono-and di-acetylated monoglycerides; or mixtures thereof.

Preferably, the gelator system of the present invention consisting of a blend of surfactants comprise a lipophilic surfactant which is a sorbitan fatty acid ester selected from a group comprising sorbitan monolaurate (SPAN® 20) , sorbitan monopalmitate (SPAN® 40), sorbitan monooleate (SPAN® 60), and sorbitan monostearate (SPAN® 80); and a hydrophilic surfactant which is a polyoxyethylene sorbitan fatty acid ester selected from a group comprising polyoxyethylene sorbitan monolaurate (TWEEN® 20), polyoxyethylene sorbitan monopalmitate (TWEEN® 40), polyoxyethylene sorbitan monooleate (TWEEN® 60), and polyoxyethylene sorbitan monostearate (TWEEN® 80). More preferably, the lipophilic surfactant is SPAN® 80 and the hydrophilic surfactant is TWEEN® 80.

In an embodiment of the present invention, the ratio of hydrophilic surfactant to lipophilic surfactant is about 1:20 to about 20:1.

The solvent system of the present invention comprises at least one oily component, and one or more other components selected from a group comprising but not limited to lipophilic solvents and hydrophilic solvents such as methanol, ethanol, isopropanol, triethyl citrate, acetyl butyl citrate or triacetin, ethylene glycol, propylene glycol, glycerol, polyethylene glycol, and polyethylene glycol esters.

The oily components of the solvent system is selected from but not limited to natural oils, mono-, di-, or triglyceride esters of oils selected from a group consisting of medium chain triglycerides, oleic acid, ethyl oleate, ethyl caprylate, ethyl butyrate, isopropyl myristate, soyabean oil, canola oil or their mono-and di-glycerides, aluminium monomonostearate, aluminium dimonostearate, aluminium trimonostearate, microcrystalline wax, petroleum wax and mixtures, used either alone or in combination thereof. Preferably, the at least one oily component of the solvent system is a medium chain triglyceride.

In another embodiment of the pharmaceutical composition of the present invention, the aqueous phase comprises water, aliphatic or aromatic alcohols, glycols, or mixtures thereof

The lipophilic solvents include triethyl citrate, acetyl butyl citrate or triacetin, triglyceride selected from but not limited to the group comprising of vegetable oils, fish oils, animal fats, hydrogenated vegetable oils, partially hydrogenated vegetable oils, synthetic triglycerides, modified triglycerides, fractionated triglycerides, and mixtures, used either alone or in combination thereof.

Hydrophilic solvents are selected from but not limited to a group consisting of water, glycols, for example propylene glycol, butylene glycol, hexylene glycol, ethylene glycol and the polyethylene glycols; and mixtures, used either alone or in combination thereof.

In an embodiment of the present invention, the solvent system comprises of at least one oily component(s) and/or at least one lipophilic solvent(s), and optionally hydrophilic solvent(s); the said composition may further contain from 1% to 30% by weight of aqueous phase relative to the total weight of the composition.

In an embodiment, the composition of the present invention optionally comprises a stabilizing agent(s), wherein the stabilizing agent is a natural, synthetic, or semisynthetic polymer which act as structure former and stabilizer in the topical formulations which range from an emulsion, cream, lotion or gel in their consistency and architecture.

The stabilizing agent(s) useful in the present invention are selected from a group of natural and synthetic polymers and carbohydrates such as chitosan, alginates, carrageenan, cellulose derivatives, pectin, starch, xanthan gum, albumin, alginate, gelatin, acacia, cellulose dextran, hydroxyethyl cellulose, hydroxypropyl cellulose, hydroxypropylmethyl cellulose, colloidal silicon dioxide, hyaluronic acid, carboxyethyl cellulose, carboxymethyl cellulose, Poloxamer (polyethylene-propylene glycol copolymer), Cabopol (carbomer), Acrylic acid based polymers and derivatives thereof. Preferably the stabilizing agent of the present invention is Poloxamer.

In yet another embodiment, the stabilizer is added either in the oily phase or in aqueous phase or added as an aqueous solution up to about a concentration ranging from 0.1% to 20% of the total weight of the composition.

In an embodiment of the present invention, the other pharmaceutically acceptable excipients are selected from but not limited to the group comprising of preservatives, formulation aids, antioxidants, diluents, pH adjusting agents, buffering agents, tonicity modifiers, colorants, and the like, or mixtures thereof.

In an embodiment of the present invention, the preservative and antioxidants are selected from a group comprising of parabens such as methylparaben sodium, propylparaben sodium, benzalkonium chloride, benzyl alcohol, sodium metabisulfite, butylated hydroxytoluene, butylated hydroxyanisole, sulphur compounds, and the like or mixtures thereof.

In an embodiment of the present invention, the formulation aid may be selected from a group comprising of poloxamer, carbopol, cellulose polymers, acrylic acid based polymer and the like, or mixtures thereof.

In an embodiment of the present invention, the formulation aid may be selected from a group comprising of citric acid, tartaric acid, and the like.

In an embodiment, the compositions of the present invention are in the form of a cream, gel, jelly, lotion, ointment, topical solution or the like, preferably in the form of a cream or gel.

In another embodiment, the compositions of the present invention is meant for highly localized topical administration for hydrophobic and/or amphiphilic active ingredient(s), including but not limited to antibacterial, antifungal, anti-parasite, anti-mycotic, antibiotic, anti-inflammatory, analgesic (narcotic and non-narcotic), anti-septic, disinfectant, anti-psoriatic, anti-eczema, anti-ageing, anti-histaminic, anti-pruritic, keratolytic, anti-seborrheic, gluco-corticoid, steroid, immunomodulators, muscle relaxant, anti-viral, anesthetic, anti-allergic, or their salts, esters, hydrates or derivatives, used either alone or in combination thereof.

In a still further embodiment, the analgesic and/or anti-inflammatory agent selected from but not limited to a group comprising of nimesulide, acetaminophen, acetanilide, acetylsalicylates, acetylsalicylic acid, alminoprofen, aspirin, benoxaprofen, carbamazepine, diflunisal, enfenamic acid, etodolac, fenoprofen, flufenamic acid, flurbiprofen, diclofenac, ibufenac, piroxicam, indomethacin, indoprofen, ketoprofen, ketorolac, miroprofen, morpholine salicylate, naproxen, phenacetin, phenyl salicylate, quinine salicylate, salicylamide, salicylic acid, salicylates and their derivatives, tenoxicam, tolfenamic acid, tramadol etc., or their salts, esters, hydrates or derivatives thereof.

Surprisingly, the present inventors have found that compositions comprising of a combination of a lipophilic and hydrophilic surfactant can gelate a combination of oily and lipophilic solvents (collectively referred to as ‘solvent system’) and incorporate sufficient amount of aqueous phase without changing the lipid microenvironment and gel architecture. Use of these compositions result in an enhanced localization of hydrophobic and/or amphiphilic active ingredient(s) essentially for the treatment of microbial and/or fungal infections of the skin layers, or autoimmune or hormonal disorders.

In the present invention, the gelator components (combination of surfactants) provide gelation of the solvent system and thus form a three dimensional network. This is due to the fact that surfactant molecules have a tendency to associate in solvent environment leading to the formation of aggregates. These further associate with others through contact points, and thus three-dimensional networks are established, which immobilize the solvent system and acts as gel. The addition of aqueous components do not generally break these tubular and torroid structures and furthermore, the stabilizing agent(s) emulsify the excess oil, which has not been gelated during the process of gelation. This also provides a cosmetic appearance to the composition. Further, this highly lipophilic microenvironment on interaction with skin lipids is intended to form a depot within the skin layers through which the entrapped hydrophobic drug could be released over an extended period of time in a localized area.

In a preferred embodiment, the therapeutic agent(s) present in the pharmaceutical compositions of the invention are about 0.1% to about 10% by weight, based on the total weight of the pharmaceutical composition.

In yet another embodiment, the present invention provides process for the preparation of a pharmaceutical composition for topical administration providing an enhanced localization of active ingredient comprising of at least one active ingredient, its salts, esters, hydrates or derivatives; a gelator system consisting of a blend of surfactants, a solvent system comprising at least one oily component; an aqueous phase comprising one or more stabilizing agent; and optionally other pharmaceutically acceptable excipients; wherein the blend of surfactants act as gelators of the oily component present in the solvent system forming a three dimensional network which immobilize the solvent system characterized such that the surfactant gelled oily phase can accommodate the aqueous phase without changing the lipid microenvironment and gel architecture of the composition.

In another embodiment, the process of preparation of compositions of the present invention comprises the preparation of an oily phase by mixing the ingredients under temperature and stirring followed by incorporation of the active ingredient(s) with stirring; preparation of an aqueous phase by mixing the ingredients under temperature and stirring; and mixing both the oily phase and the aqueous phase under temperature and stirring to obtain the desired product.

The present invention also provides methods for the management/treatment of fungal, bacterial or microbial infections, inflammations, autoimmune conditions, or hormonal disorders comprising administering to a subject in need of such treatment a pharmaceutically effective amount of a pharmaceutical composition as described herein.

In order to illustrate embodiments of the present invention, the following examples are provided. However, these examples do not intent to limit the scope of the invention.

### EXAMPLES

### Example 1

| S. No. | Ingredients | Quantity (mg/g) |
|---|---|---|
| 1. | Terbinafine hydrochloride | 10.00 |
| 2. | Sorbitan monostearate | 195.00 |
| 3. | Polysorbate 20 | 21.50 |
| 4. | Medium chain triglyceride | 41.85 |
| 5. | Poloxamer 188 aqueous (7% w/w) solution | 34.00 |
| 6. | Benzyl alcohol | 10.00 |
| 7. | Sodium metabisulphite | 5.00 |
| 8. | Purified water | q.s. to 1.00 g |

The topical formulation was prepared as follows.

Predetermined weighed amounts of Sorbitan monostearate, Polysorbate 20, Medium chain triglyceride and Benzyl alcohol were taken. The contents were heated with continuous stirring in a constant temperature water bath while maintaining the temperature of the mass at 60-65° C. Terbinafine hydrochloride was added in the melt, while stirring until homogenous mixing was achieved. An aqueous phase was prepared. A predetermined weighed amount of Poloxamer 188 was mixed with purified water (7% w/w). To this was added Sodium metabisulphite in prescribed quantity. The mixture was stirred and then heated while maintaining the temperature of the mass at 60-65° C. The oily phase and aqueous phase were maintained at 60-65° C. and bulk of aqueous phase was added to oily phase maintaining the similar temperature (60-65° C.) with continuous stirring to obtain the desired product.

### Example 2

| S. No. | Ingredients | Quantity (mg/g) |
|---|---|---|
| 1. | Terbinafine hydrochloride | 10.00 |
| 2. | Sorbitan monostearate | 195.00 |
| 3. | Polysorbate 20 | 21.50 |
| 4. | Medium chain triglyceride | 318.50 |
| 5. | Carbopol 971P aqueous (2% w/w) solution | 250.00 |
| 6. | Benzyl alcohol | 10.00 |
| 7. | Sodium metabisulphite | 5.00 |
| 8. | Triethanolamine | 100.00 |
| 9. | Purified water | q.s. to 1.00 g |

The topical formulation was prepared as follows.

Predetermined weighed amounts of Sorbitan monostearate, Polysorbate 20, Medium chain triglyceride and Benzyl alcohol were taken. The contents were heated with continuous stirring in a constant temperature water bath while maintaining the temperature of the mass at 60-65° C. Terbinafine hydrochloride was added in the melt, while stirring until homogenous mixing was achieved. An aqueous phase was prepared. A predetermined weighed amount of Carbopol 971P and Triethanolamine was mixed with purified water (2% w/w). To this was added Sodium metabisulphite in prescribed quantity and the mixture was stirred while maintaining the temperature of the mass at 60-65° C. The oily phase and aqueous phase were maintained at 60-65° C. and bulk of aqueous phase was added to oily phase maintaining the similar temperature (60-65° C.) with continuous stirring to obtain the desired product.

### Example 3

| S. No. | Ingredients | Quantity (mg/g) |
|---|---|---|
| 1. | Butenafine hydrochloride | 10.00 |
| 2. | Sorbitan monostearate | 195.00 |
| 3. | Polysorbate 20 | 21.50 |
| 4. | Medium chain triglyceride | 318.50 |
| 5. | Sodium alginate aqueous (2% w/w) solution | 250.00 |
| 6. | Benzyl alcohol | 10.00 |
| 7. | Sodium metabisulphite | 5.00 |
| 8. | Triethanolamine | 100.00 |
| 9. | Purified water | q.s. to 1.00 g |

The topical formulation was prepared as follows.

Predetermined weighed amounts of Sorbitan monostearate, Polysorbate 20, Medium chain triglyceride and Benzyl alcohol were taken. The contents were heated with continuous stirring in a constant temperature water bath while maintaining the temperature of the mass at 60-65° C. Butenafine hydrochloride was added in the melt, while stirring until homogenous mixing was achieved. An aqueous phase was prepared. A predetermined weighed amount of Sodium alginate and Triethanolamine was mixed with purified water (2% w/w). To this was added Sodium metabisulphite in prescribed quantity and the mixture was stirred while maintaining the temperature of the mass at 60-65° C. The oily phase and aqueous phase were maintained at 60-65° C. and bulk of aqueous phase was added to oily phase maintaining the similar temperature (60-65° C.) with continuous stirring to obtain the desired product.

### Example 4

| S. No. | Ingredients | Quantity (mg/g) |
|---|---|---|
| 1. | Terbinafine hydrochloride | 10.00 |
| 2. | Glyceryl monomonostearate | 195.00 |
| 3. | Polysorbate 20 | 21.50 |
| 4. | Medium chain triglyceride | 318.50 |
| 5. | Sodium alginate aqueous (2% w/w) solution | 250.00 |
| 6. | Benzyl alcohol | 10.00 |
| 7. | Sodium metabisulphite | 5.00 |
| 8. | Triethanolamine | 100.00 |
| 9. | Purified water | q.s. to 1.00 g |

The topical formulation was prepared as follows.

Predetermined weighed amounts of Glyceryl monomonostearate, Polysorbate 20, Medium chain triglyceride and Benzyl alcohol were taken. The contents were heated with continuous stirring in a constant temperature water bath while maintaining the temperature of the mass at 60-65° C. Terbinafine Hydrochloride was added in the melt, while stirring until homogenous mixing was achieved. An aqueous phase was prepared. A predetermined weighed amount of sodium alginate and triethanolamine were mixed with purified water (2% w/w). To this was added Sodium metabisulphite in prescribed quantity and the mixture was stirred while maintaining the temperature of the mass at 60-65° C. The oily phase and aqueous phase were maintained at 60-65° C. and bulk of aqueous phase was added to oily phase maintaining the similar temperature (60-65° C.) with continuous stirring to obtain the desired product.

### Example 5

| S. No. | Ingredients | Quantity (mg/g) | |
|---|---|---|---|
| 1. | Terbinafine hydrochloride | 10.00 | |
| 2. | Glyceryl monomonostearate | 19.50 | |
| 3. | Polysorbate 20 | 21.50 | |
| 4. | Isopropyl myristate | 318.50 | |
| 5. | Poloxamer 188 aqueous (10% w/w) solution | 0.250 | |
| 6. | Benzyl alcohol | 10.00 | |
| 7. | Sodium metabisulphite | 5.00 | |
| 8. | Triethanolamine | 100.00 | |
| 9. | Purified water | q.s. to 1.00 | g |

The topical formulation was prepared as follows.

Predetermined weighed amounts of Glyceryl monomonostearate, Polysorbate 20, Isopropyl myristate and Benzyl alcohol were taken. The contents were heated with continuous stirring in a constant temperature water bath while maintaining the temperature of the mass at 60-65° C. Terbinafine Hydrochloride was added in the melt, while stirring until homogenous mixing was achieved. An aqueous phase was prepared. A predetermined weighed amount of Poloxamer 188 and triethanolamine were mixed with purified water (10% w/w). To this was added Sodium metabisulphite in prescribed quantity and the mixture was stirred while maintaining the temperature of the mass at 60-65° C. The oily phase and aqueous phase were maintained at 60-65° C. and bulk of aqueous phase was added to oily phase maintaining the similar temperature (60-65° C.) with continuous stirring to obtain the desired product.

### Example 6

| S. No. | Ingredients | Quantity (mg/g) |
|---|---|---|
| 1. | Terbinafine hydrochloride | 10.00 |
| 2. | Sorbitan monostearate | 250.00 |
| 3. | Polysorbate 20 | 25.00 |
| 4. | Medium chain triglyceride | 250.00 |
| 5. | Isopropyl myristate | 255.00 |
| 6. | Propylene glycol | 200.00 |
| 7. | Benzyl alcohol | 10.00 |

The topical formulation was prepared as follows.

Predetermined weighed amounts of Sorbitan monostearate, Polysorbate 20, Medium chain triglyceride, Propylene glycol, Isopropyl myristate and Benzyl alcohol were taken. The contents were heated with continuous stirring in a constant temperature water bath while maintaining the temperature of the mass at 60-65° C. Terbinafine hydrochloride was added in the melt, while stirring until homogenous mixing was achieved. The off-white to cream-colored formulation thus obtained can be stored in tightly closed HDPE container.

### Example 7

| S. No. | Ingredients | Quantity (mg/g) |
|---|---|---|
| 1. | Terbinafine hydrochloride | 10.00 |
| 2. | Sorbitan monostearate | 250.00 |
| 3. | Polysorbate 20 | 25.00 |
| 4. | Medium chain triglyceride | 250.00 |
| 5. | Propylene glycol | 75.00 |
| 6. | Chitosan | 40.00 |
| 7. | Citric acid | 90.00 |
| 8. | Benzyl alcohol | 10.00 |
| 9. | Purified water | 250.00 |

The topical formulation was prepared as follows.

An oily phase was prepared first. Predetermined weighed amounts of Sorbitan monostearate, Polysorbate 20, Medium chain triglyceride, Propylene glycol and Benzyl alcohol are taken; the liquid ingredients were passed through nylon cloth and transferred to a jacketed S.S. container. The solid ingredients were added to the contents of the S.S. container and mixed. This mixture was heated with continuous stirring by circulating hot water in the jacket while maintaining the temperature of the mass at 60-65° C. Terbinafine hydrochloride was added in the above melt, while stirring until homogenous mixing was achieved. An aqueous phase was then prepared. Predetermined weighed amounts of Chitosan and Citric acid were mixed with sufficient purified water and the mixture was heated with continuous stirring while maintaining the temperature of the mass at 60-65° C. The oily phase and aqueous phase were maintained at 60-65° C. and bulk of aqueous phase was added to oily phase maintaining the similar temperature (60-65° C.) with continuous stirring to obtain the desired formulation.

### Example 8

| S. No. | Ingredients | Quantity (mg/g) |
|---|---|---|
| 1. | Terbinafine hydrochloride | 10.00 |
| 2. | Nimesulide | 10.00 |
| 2. | Glyceryl monomonostearate | 250.00 |
| 3. | Polysorbate 20 | 50.00 |
| 4. | Propylene glycol | 320.00 |
| 5. | Isopropyl myristate | 350.00 |
| 6. | Benzyl alcohol | 10.00 |

The topical formulation was prepared as follows.

Predetermined weighed amounts of Glyceryl monomonostearate, Polysorbate 20, Isopropyl myristate, Propylene glycol and Benzyl alcohol were taken. The contents were heated with continuous stirring while maintaining the temperature of the mass at 60-65° C. Terbinafine hydrochloride and Nimesulide were added in melt, while stirring until homogenous mixing was achieved. The off-white to cream-colored formulation thus obtained was stored in tightly closed HDPE container.

### Example 9

| S. No. | Ingredients | Quantity (mg/g) |
|---|---|---|
| 1. | Clotrimazole | 10.00 |
| 2. | Polyethylene glycol dimonostearate | 250.00 |
| 3. | Polysorbate 20 | 25.00 |
| 4. | Mineral oil | 250.00 |
| 5. | Chitosan | 40.00 |
| 6. | Citric acid | 80.00 |
| 7. | Benzyl alcohol | 10.00 |
| 8. | Purified water | 335.00 |

The topical formulation was prepared as follows.

An oily phase was prepared first. Predetermined weighed amounts of Polyethylene glycol dimonostearate, Polysorbate 20, Medium chain triglyceride, Mineral oil and Benzyl alcohol were taken; the liquid ingredients were passed through nylon cloth and transferred it to a jacketed S.S. container. The solid ingredients were added to the contents of the S.S. container and mixed. This mixture was heated with continuous stirring by circulating hot water in the jacket while maintaining the temperature of the mass at 60-65° C. Clotrimazole was added in the above melt, while stirring until homogenous mixing was achieved. An aqueous phase was then prepared. Predetermined weighed amounts of Chitosan and Citric acid were mixed with sufficient purified water and the mixture was heated with continuous stirring while maintaining the temperature of the mass at 60-65° C. The oily phase and aqueous phase were maintained at 60-65° C. and bulk of aqueous phase was added to oily phase maintaining the similar temperature (60-65° C.) with continuous stirring to obtain the desired product.

### Example 10

| S. No. | Ingredients | Quantity (mg/g) |
|---|---|---|
| 1. | Miconazole | 20.00 |
| 2. | Gentamycin sulphate | 10.00 |
| 3. | Polyethylene glycol dimonostearate | 250.00 |
| 4. | Polysorbate 20 | 25.00 |
| 5. | Isopropyl myristate | 250.00 |
| 6. | Chitosan | 40.00 |
| 7. | Citric Acid | 80.00 |
| 8. | Benzyl alcohol | 10.00 |
| 9. | Purified Water | 315.00 |

The topical formulation was prepared as follows.

An oily phase was prepared first. Predetermined weighed amounts of Polyethylene glycol dimonostearate, Polysorbate 20, Isopropyl myristate and Benzyl alcohol were taken; the liquid were passed ingredients through nylon cloth and transferred to a jacketed S.S. container. The solid ingredients were added to the contents of the S.S. container and mixed. This mixture was heated with continuous stirring by circulating hot water in the jacket while maintaining the temperature of the mass at 60-65° C. Miconazole and Gentamycin sulphate were added in the above melt, while stirring until homogenous mixing was achieved. An aqueous phase was prepared. Predetermined weighed amounts of Chitosan and Citric acid were mixed with sufficient purified water and the mixture was heated with continuous stirring while maintaining the temperature of the mass at 60-65° C. The oily phase and aqueous phase were maintained at 60-65° C. and bulk of aqueous phase was added to oily phase maintaining the similar temperature (60-65° C.) with continuous stirring to obtain the desired product.

### Example 11

| S. No. | Ingredients | Quantity (mg/g) |
|---|---|---|
| 1. | Sertaconazole | 10.00 |
| 2. | Sorbitan monostearate | 250.00 |
| 3. | Polysorbate 20 | 25.00 |
| 4. | Medium chain triglyceride | 250.00 |
| 5. | Propylene glycol | 75.00 |
| 6. | Chitosan | 40.00 |
| 7. | Citric acid | 90.00 |
| 8. | Benzyl alcohol | 10.00 |
| 9. | Purified water | 250.00 |

The topical formulation was prepared as follows.

An oily phase was prepared first. Predetermined weighed amounts of Sorbitan monostearate, Polysorbate 20, Medium chain triglyceride, Propylene glycol and Benzyl alcohol are taken; the liquid ingredients were passed through nylon cloth and transferred to a jacketed S.S. container. The solid ingredients were added to the contents of the S.S. container and mixed. This mixture was heated with continuous stirring by circulating hot water in the jacket while maintaining the temperature of the mass at 60-65° C. Sertaconazole was added in the above melt, while stirring until homogenous mixing was achieved. An aqueous phase was then prepared. Predetermined weighed amounts of Chitosan and Citric acid were mixed with sufficient purified water and the mixture was heated with continuous stirring while maintaining the temperature of the mass at 60-65° C. The oily phase and aqueous phase were maintained at 60-65° C. and bulk of aqueous phase was added to oily phase maintaining the similar temperature (60-65° C.) with continuous stirring to obtain the desired formulation.

### Example 12

| S. | | | |
|---|---|---|---|
| No. | Ingredients | Quantity (mg/g) | |
| 1. | Terbinafine hydrochloride | 10.00 | |
| 2. | Methanol | 20.00 | |
| 3. | Medium chain triglyceride (Crodamol GTC/C) | 412.50 | |
| 4. | Sorbitan monostearate (SPAN-60) | 195.00 | |
| 5. | Polyoxyethylene Sorbitan monolaurate | 21.50 | |
| | (Polysorbate 20) | | |
| 6. | Butylated hydroxytoluene (BHT) | 0.90 | |
| 7. | Butylated hydroxyanisole (BHA) | 0.01 | |
| 8. | Poloxamer 118 | 26.80 | |
| 9. | Triethanolamine | 0.75 | |
| 10. | Benzyl alcohol | 10.00 | |
| 11. | Purified water | q.s. to 1.00 | g |

The topical formulation was prepared as follows.

An oily phase was prepared first. Predetermined weighed amounts of Sorbitan monostearate, Polysorbate 20, Medium chain triglyceride, Butylated hydroxytoluene and Butylated hydroxyanisole were taken; the liquid ingredients were passed through nylon cloth and transferred to a jacketed S.S. container. The solid ingredients were added to the contents of the S.S. container and mixed. This mixture was heated with continuous stirring by circulating hot water in the jacket while maintaining the temperature of the mass at 50-55° C. Terbinafine hydrochloride was dissolved in methanol and added in the above melt, while stirring until homogenous mixing was achieved. An aqueous phase was then prepared. Predetermined weighed amounts of Poloxamer and Triethanolamine was mixed with sufficient purified water and Benzyl alcohol was added and the mixture was heated with continuous stirring while maintaining the temperature of the mass at 50-55° C. The oily phase and aqueous phase were maintained at 60-65° C. and bulk of aqueous phase was added to oily phase maintaining the similar temperature (60-65° C.) with continuous stirring to obtain the desired formulation.

### Example 13

| S. | | | |
|---|---|---|---|
| No. | Ingredients | Quantity (mg/g) | |
| 1. | Terbinafine hydrochloride | 10.00 | |
| 2. | Methanol | 20.00 | |
| 3. | Medium chain triglyceride (Crodamol GTC/C) | 412.50 | |
| 4. | Sorbitan monostearate (SPAN-60) | 195.00 | |
| 5. | Polyoxyethylene Sorbitan monolaurate | 21.50 | |
| | (Polysorbate 20) | | |
| 6. | Butylated hydroxytoluene (BHT) | 0.90 | |
| 7. | Butylated hydroxyanisole (BHA) | 0.01 | |
| 8. | Poloxamer 118 | 26.80 | |
| 9. | Triethanolamine | 0.75 | |
| 10. | Methylparaben sodium | 1.80 | |
| 11 | Propylparaben sodium | 0.20 | |
| 12. | Propylene glycol | 10.00 | |
| 13. | Purified water | q.s. to 1.00 | g |

The topical formulation was prepared as follows.

An oily phase was prepared first. Predetermined weighed amounts of Sorbitan monostearate, Polysorbate 20, Medium chain triglyceride, Propylparaben sodium, Butylated hydroxytoluene and Butylated hydroxyanisole were taken; the liquid ingredients were passed through nylon cloth and transferred to a jacketed S.S. container. The solid ingredients were added to the contents of the S.S. container and mixed. This mixture was heated with continuous stirring by circulating hot water in the jacket while maintaining the temperature of the mass at 50-55° C. Terbinafine hydrochloride was dissolved in methanol and added in the above melt, while stirring until homogenous mixing was achieved. An aqueous phase was then prepared. Predetermined weighed amounts of Poloxamer and Methylparaben sodium were mixed with sufficient purified water and Propylene glycol was added and the mixture was heated with continuous stirring while maintaining the temperature of the mass at 50-55° C. The oily phase and aqueous phase were maintained at 60-65° C. and bulk of aqueous phase was added to oily phase maintaining the similar temperature (60-65° C.) with continuous stirring followed by the addition of Benzyl alcohol to obtain the desired formulation.

### Example 14

| S. | | | |
|---|---|---|---|
| No. | Ingredients | Quantity (mg/g) | |
| 1. | Tacrolimus | 0.30 | |
| 2. | Methanol | 20.00 | |
| 3. | Medium chain triglyceride (Crodamol GTC/C) | 422.20 | |
| 4. | Sorbitan monostearate (SPAN-60) | 195.00 | |
| 5. | Polyoxyethylene Sorbitan monolaurate | 21.50 | |
| | (Polysorbate 20) | | |
| 6. | Butylated hydroxytoluene (BHT) | 0.90 | |
| 7. | Butylated hydroxyanisole (BHA) | 0.01 | |
| 8. | Poloxamer 118 | 26.80 | |
| 9. | Triethanolamine | 0.75 | |
| 10. | Benzyl alcohol | 10.00 | |
| 11. | Purified water | q.s. to 1.00 | g |

The topical formulation was prepared as follows.

An oily phase was prepared first. Predetermined weighed amounts of Sorbitan monostearate, Polysorbate 20, Medium chain triglyceride, Butylated hydroxytoluene and Butylated hydroxyanisole were taken; the liquid ingredients were passed through nylon cloth and transferred to a jacketed S.S. container. The solid ingredients were added to the contents of the S.S. container and mixed. This mixture was heated with continuous stirring by circulating hot water in the jacket while maintaining the temperature of the mass at 50-55° C. Tacrolimus was dissolved in methanol and added in the above melt, while stirring until homogenous mixing was achieved. An aqueous phase was then prepared. Predetermined weighed amounts of Poloxamer and Triethanolamine were mixed with sufficient purified water and benzyl alcohol was added and the mixture was heated with continuous stirring while maintaining the temperature of the mass at 50-55° C. The oily phase and aqueous phase were maintained at 60-65° C. and bulk of aqueous phase was added to oily phase maintaining the similar temperature (60-65° C.) with continuous stirring to obtain the desired formulation.

### Example 15

| S. | | | |
|---|---|---|---|
| No. | Ingredients | Quantity (mg/g) | |
| 1. | Tacrolimus | 1.00 | |
| 2. | Ethanol | 20.00 | |
| 3. | Medium chain triglyceride (Crodamol GTC/C) | 399.00 | |
| 4. | Sorbitan monostearate (SPAN-60) | 195.00 | |
| 5. | Polyoxyethylene Sorbitan monolaurate | 21.40 | |
| | (Polysorbate 20) | | |
| 6. | Butylated hydroxytoluene (BHT) | 0.90 | |
| 7. | Butylated hydroxyanisole (BHA) | 0.01 | |
| 8. | Poloxamer 118 | 23.00 | |
| 9. | Methylparaben sodium | 1.80 | |
| 10 | Propylparaben sodium | 0.20 | |
| 11. | Propylene glycol | 51.40 | |
| 12. | Tartaric acid | 1.00 | |
| 13. | Purified water | q.s. to 1.00 | g |

The topical formulation was prepared as follows.

An oily phase was prepared first. Predetermined weighed amounts of Sorbitan monostearate, Polysorbate 20, Medium chain triglyceride, Propylparaben sodium, Butylated hydroxytoluene and Butylated hydroxyanisole were taken; the liquid ingredients were passed through nylon cloth and transferred to a jacketed S.S. container. The solid ingredients were added to the contents of the S.S. container and mixed. This mixture was heated with continuous stirring by circulating hot water in the jacket while maintaining the temperature of the mass at 50-55° C. Tacrolimus was dissolved in methanol and added in the above melt, while stirring until homogenous mixing was achieved. An aqueous phase was then prepared. Predetermined weighed amounts of Poloxamer and Methylparaben sodium were mixed with sufficient purified water and propylene glycol was added and the mixture was heated with continuous stirring while maintaining the temperature of the mass at 50-55° C. The oily phase and aqueous phase were maintained at 60-65° C. and bulk of aqueous phase was added to oily phase maintaining the similar temperature (60-65° C.) with continuous stirring followed by the addition of Benzyl alcohol to obtain the desired formulation.

### Example 16

| S. | | | |
|---|---|---|---|
| No. | Ingredients | Quantity (mg/g) | |
| 1. | Cyclosporine | 50.00 | |
| 2. | Methanol | 20.00 | |
| 3. | Medium chain triglyceride (Crodamol GTC/C) | 372.50 | |
| 4. | Sorbitan monostearate (SPAN-60) | 195.00 | |
| 5. | Polyoxyethylene Sorbitan monolaurate | 21.50 | |
| | (Polysorbate 20) | | |
| 6. | Butylated hydroxytoluene (BHT) | 0.90 | |
| 7. | Butylated hydroxyanisole (BHA) | 0.01 | |
| 8. | Poloxamer 118 | 26.80 | |
| 9. | Triethanolamine | 0.75 | |
| 10. | Benzyl alcohol | 10.00 | |
| 11. | Purified water | q.s. to 1.00 | g |

The topical formulation was prepared as follows.

An oily phase was prepared first. Predetermined weighed amounts of Sorbitan monostearate, Polysorbate 20, Medium chain triglyceride, Butylated hydroxytoluene and Butylated hydroxyanisole were taken; the liquid ingredients were passed through nylon cloth and transferred to a jacketed S.S. container. The solid ingredients were added to the contents of the S.S. container and mixed. This mixture was heated with continuous stirring by circulating hot water in the jacket while maintaining the temperature of the mass at 50-55° C. Cyclosporine was dissolved in methanol and added in the above melt, while stirring until homogenous mixing was achieved. An aqueous phase was then prepared. Predetermined weighed amounts of Poloxamer and Triethanolamine were mixed with sufficient purified water and Benzyl alcohol was added and the mixture was heated with continuous stirring while maintaining the temperature of the mass at 50-55° C. The oily phase and aqueous phase were maintained at 60-65° C. and bulk of aqueous phase was added to oily phase maintaining the similar temperature (60-65° C.) with continuous stirring to obtain the desired formulation.

### Example 17

| S. | | | |
|---|---|---|---|
| No. | Ingredients | Quantity (mg/g) | |
| 1. | Cyclosporine | 80.00 | |
| 2. | Ethanol | 20.00 | |
| 3. | Medium chain triglyceride (Crodamol GTC/C) | 320.00 | |
| 4. | Sorbitan monostearate (SPAN-60) | 195.00 | |
| 5. | Polyoxyethylene Sorbitan monolaurate | 21.40 | |
| | (Polysorbate 20) | | |
| 6. | Butylated hydroxytoluene (BHT) | 0.90 | |
| 7. | Butylated hydroxyanisole (BHA) | 0.01 | |
| 8. | Poloxamer 118 | 23.00 | |
| 9. | Methylparaben sodium | 1.80 | |
| 10 | Propylparaben sodium | 0.20 | |
| 11. | Propylene glycol | 51.40 | |
| 12. | Tartaric acid | 1.00 | |
| 13. | Purified water | q.s. to 1.00 | g |

The topical formulation was prepared as follows.

An oily phase was prepared first. Predetermined weighed amounts of Sorbitan monostearate, Polysorbate 20, Medium chain triglyceride, Propylparaben sodium, Butylated hydroxytoluene and Butylated hydroxyanisole were taken; the liquid ingredients were passed through nylon cloth and transferred to a jacketed S.S. container. The solid ingredients were added to the contents of the S.S. container and mixed. This mixture was heated with continuous stirring by circulating hot water in the jacket while maintaining the temperature of the mass at 50-55° C. Cyclosporine was dissolved in methanol and added in the above melt, while stirring until homogenous mixing was achieved. An aqueous phase was then prepared. Predetermined weighed amounts of Poloxamer and Methylparaben sodium were mixed with sufficient purified water and Propylene glycol was added and the mixture was heated with continuous stirring while maintaining the temperature of the mass at 50-55° C. The oily phase and aqueous phase were maintained at 60-65° C. and bulk of aqueous phase was added to oily phase maintaining the similar temperature (60-65° C.) with continuous stirring followed by the addition of benzyl alcohol to obtain the desired formulation.

Dermatopharmacokineic Study

The dermatopharmacokinetic (DPK) studies in the present invention are used to mimic clinical trials as a means of documenting bioavailability and equivalence of topical drug products. For the therapeutic class of anti-fungal drugs, the stratum corneum itself is the site of action. For example, in fungal infections of the skin, the fungi reside in the stratum corneum and therefore DPK measurement of an antifungal drug in the stratum corneum represents direct measurement of drug concentration at the site of action. No better assay of bioequivalence can be envisioned for this class of compounds than direct assay of the target tissue. The “Tape stripping” method used is capable of demonstrating differences of stratum corneum (SC) localization of the said invention over competitor products. This is determined by applying different compositions of the said invention to the skin surface for a specified exposure time, adhesive films are placed on the treated skin and are stripped off again after a certain application time and analysis of the localized amount in stratum corneum using validated analytical method to measure the localization index in the stratum corneum per unit surface of applied area.

Dermatopharmacokinetic (DPK) study was done to determine the comparative efficacy of Terbinafine HCl topical formulations of Innovator product (Lamisil®, herein referred to as INV) and Panacea Biotec Ltd. (DDR-FRD-F1, herein referred to as PBL). The composition described in Example 2 above has been coded as DDR-FRD-F1 and used for the said study. The assay values of the compositions used for the study were as follows: Lamisil® contained 0.099 mg of Terbinafine HCl per mg of cream formulation and DDR-FRD-F1 (coded for Example 2) contained 0.090 mg of Terbinafine HCl per mg of cream formulation.

Tape stripping experiment was performed following the drafted guidance of US FDA (Guidance for Industry: Topical dermatological drug product NDAs and ANDAs- In vitro bioavailability, bioequivalence, in vitro release and associated studies). The general test procedure in the mentioned study is as follows: First, the hair of the experimental animal (guinea pig) is removed by plucking (preferably) and then the animals are exposed in a conditioned room maintained at 20° C. with 60% RH. This condition has to be maintained throughout the experimental period. The dorsal side of the guinea pigs (2×2.5 cm²) is marked on left and right dorsal sites. Control is run simultaneously to check baseline reading. About 65.0 to 125.0 mg of the formulations (1% topical creams i.e. 100.0 mg formulations contain 1 mg of active drug, Terbinafine hydrochloride) were applied to the stratum corneum of 5 guinea pigs (N=5 denoted as N1, N2, N3, N4 and N5). A non-occluding protective guard is placed to cover the application area (non-occluding aluminum foil is used). The excess formulation is removed after 15 minutes from the application site by wiping three times lightly with a cotton swab. The initial and final weight of the cotton swab is measured to precisely monitor applied amount per square meter of the skin. After appropriate time intervals, the samples are collected following tape stripping using adhesive tape. Transpore™ tape (Model 1527-1, surface area 2.5 cm², 3M) is used as an adhesive tape. The adhesive tape is applied using uniform pressure and removed at different time intervals using constant peel off force. The duration of the study was 24 hours at the following intervals: 0.5, 1.0, 3.0, 6.0, 12.0 and 24.0 hours. A blunt ended forceps is used to apply individual adhesive tap with a constant pressure, by the same investigator every time. Both test and reference products are applied on the same side to counterbalance the inter-subject variation.

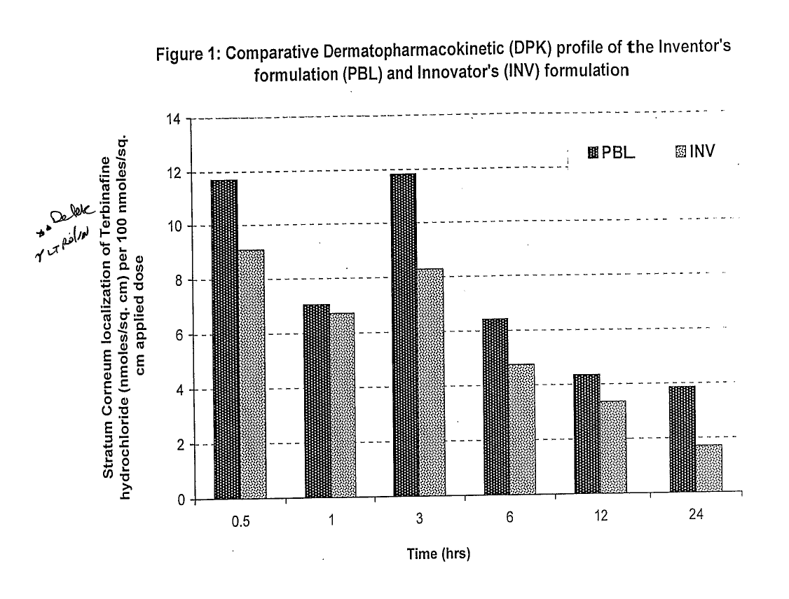

The procedure was repeated for each site at designated time points. The drug is extracted from the combined eight tape stripping and the concentration is determined using a validated analytical method. The first two tape strips are removed and not included in the analytical method validation (to accommodate residual product contamination). Further 8 tape strips are taken and pooled for each time interval and analyzed using validated method of estimation for Terbinafine hydrochloride. Tape stripping samples are stored in 10 ml polypropylene tube with 7.0 ml of 80:20 v/v acetonitrile and TEA (0.72 mM) at pH 2.5 and subjected to agitation for 16 h. In case of delay, samples are stored at −70° C. until processed. Supernatant is passed through 0.45 μm filter and subjected to validated analytical HPLC method. The results of the study are expressed as concentration of drug (nmoles) calculated to be in stratum corneum (SC) per cm² of the applied area (i.e. calculation for 100 nmol per cm² of applied cream). The results of the study are presented in tables 1-3 and in FIG. 1, as mentioned below.

Table 1: Calculation for drug localization of Inventor's formulation (PBL) in stratum corneum

Table 2: Calculation for drug localization of Innovator's formulation (INV) in stratum corneum

Table 3: Comparative efficacy of Inventor's formulation (PBL) over Inventor's formulation (INV) from Dermatopharmacokinetic (DPK) studies

FIG. 1: Comparative Dermatopharmacokinetic (DPK) profile of the Inventor's formulation (PBL) and Innovator's (INV) formulation

The results of dermatopharmacokinetic study showed a significant increase in localization of Terbinafine HCl on the skin (stratum corneum), and hence improved efficacy of the composition of the present invention over the Innovator product (Lamisil®).

TABLE 1
Calculation for drug localization of Inventor's formulation
(PBL) in stratum corneum

| | | | Equilibrated nmoles/cm² |
|---|---|---|---|
| | | nmoles/cm² | drug in SC |
| Application | nmoles/cm² | (drug) | per 100 nmoles/cm² |
| codes | (drug) applied | In SC | drug applied |
| PBL-0.5-N1 | 551.93 | 38.44 | 6.964 |
| PBL-0.5-N2 | 485.61 | 69.55 | 14.32 |
| PBL-0.5-N3 | 419.84 | 30.43 | 7.247 |
| PBL-0.5 N4 | 564.84 | 116.20 | 20.57 |
| PBL-0.5 N5 | 421.26 | 39.75 | 9.435 |
| PBL-1.0-N1 | 543.53 | 19.73 | 3.629 |
| PBL-1.0-N2 | 549.19 | 15.56 | 2.833 |
| PBL-1.0-N3 | 565.63 | 75.78 | 13.39 |
| PBL-1.0-N4 | 555.61 | 49.04 | 8.830 |
| PBL-1.0-N5 | 604.48 | 39.53 | 6.539 |
| PBL-3.0-N1 | 307.59 | 48.75 | 15.84 |
| PBL-3.0-N2 | 429.92 | 67.82 | 15.77 |
| PBL-3.0-N3 | 585.37 | 21.15 | 3.613 |
| PBL-3.0-N4 | 512.79 | 62.49 | 12.18 |
| PBL-3.0-N5 | 457.09 | 53.5 | 11.70 |
| PBL-6.0-N1 | 593.48 | 20.07 | 3.381 |
| PBL-6.0-N2 | 533.52 | 22.17 | 4.155 |
| PBL-6.0-N3 | 665.66 | 20.49 | 3.078 |
| PBL-6.0-N4 | 446.62 | 57.19 | 12.80 |
| PBL-6.0-N5 | 474.19 | 41.10 | 8.667 |
| PBL-12.0-N1 | 590.31 | 23.76 | 4.025 |
| PBL-12.0-N2 | 721.84 | 33.06 | 4.579 |
| PBL-12.0-N3 | 624.28 | 22.22 | 3.559 |
| PBL-12.0-N4 | 501.76 | 35.18 | 7.011 |
| PBL-12.0-N5 | 523.0 | 13.23 | 2.530 |
| PBL-24.0-N1 | 534.39 | 13.41 | 2.509 |
| PBL-24.0-N2 | 380.38 | 25.17 | 6.617 |
| PBL-24.0-N3 | 556.32 | 19.39 | 3.485 |
| PBL-24.0-N4 | 464.26 | 18.62 | 4.010 |
| PBL-24.0-N5 | 420.67 | 11.15 | 2.650 |

TABLE 2
Calculation for drug localization of Innovator's formulation
(INV) in stratum corneum

| | | nmoles/cm² | Equilibrated nmoles/cm² |
|---|---|---|---|
| Application | nmoles/cm² | (drug) | drug in SC per 100 |
| codes | (drug) applied | In SC | nmoles/cm² drug applied |
| INV-0.5-N1 | 807.89 | 47.12 | 5.832 |
| INV-0.5-N2 | 783.78 | 57.72 | 7.364 |
| INV-0.5-N3 | 657.11 | 50.04 | 7.615 |
| INV-0.5 N4 | 557.32 | 100.8 | 8.010 |
| INV-0.5 N5 | 537.21 | 35.15 | 6.540 |
| INV-1.0-N1 | 752.41 | 50.04 | 6.650 |
| INV-1.0-N2 | 810.88 | 34.37 | 4.238 |
| INV-1.0-N3 | 824.15 | 43.17 | 5.238 |
| INV-1.0-N4 | 609.39 | 56.93 | 9.340 |
| INV-1.0-N5 | 619.45 | 49.98 | 8.060 |
| INV-3.0-N1 | 728.91 | 101.41 | 13.91 |
| INV-3.0-N2 | 756.01 | 64.84 | 8.576 |
| INV-3.0-N3 | 760.82 | 46.51 | 6.113 |
| INV-3.0-N4 | 455.56 | 28.39 | 6.230 |
| INV-3.0-N5 | 538.98 | 35.46 | 6.570 |
| INV-6.0-N1 | 625.56 | 25.97 | 4.151 |
| INV-6.0-N2 | 775.94 | 37.30 | 4.807 |
| INV-6.0-N3 | 680.68 | 20.58 | 3.023 |
| INV-6.0-N4 | 604.08 | 42.56 | 7.040 |
| INV-6.0-N5 | 514.73 | 24.05 | 4.670 |
| INV-12.0-N1 | 757.85 | 39.03 | 5.150 |
| INV-12.0-N2 | 723.49 | 25.62 | 3.541 |
| INV-12.0-N3 | 745.19 | 5.88 | 0.789 |
| INV-12.0-N4 | 731.38 | 29.21 | 3.990 |
| INV-12.0-N5 | 555.28 | 18.19 | 3.270 |
| INV-24.0-N1 | 756.04 | 15.07 | 1.993 |
| INV-24.0-N2 | 643.9 | 14.09 | 2.188 |
| INV-24.0-N3 | 749.41 | 2.22 | 0.296 |
| INV-24.0-N4 | 792.19 | 17.09 | 2.150 |
| INV-24.0-N5 | 693.40 | 12.94 | 1.860 |

TABLE 3
Comparative efficacy of Inventor's formulation (PBL) over Innovator's
formulation (INV) from Dermatopharmacokinetic (DPK) studies

| | SC localization (nmoles/cm²) | | | | |
|---|---|---|---|---|---|
| Application | after 100.0 nmoles/cm² applied dose | | | | |
| codes | N1* | N2* | N3* | N4* | N5* |

INNOVATOR'S PRODUCT (LAMISIL ™)

| | | | | | |
|---|---|---|---|---|---|
| INV-0.5 | 5.832 | 7.364 | 7.615 | 18.02 | 6.541 |
| INV-1.0 | 6.650 | 4.238 | 5.238 | 9.341 | 8.064 |
| INV-3.0 | 13.912 | 8.576 | 6.113 | 6.233 | 6.573 |
| INV-6.0 | 4.151 | 4.807 | 3.023 | 7.042 | 4.672 |
| INV-12.0 | 5.150 | 3.541 | 0.789 | 3.993 | 3.272 |
| INV-24.0 | 1.993 | 2.188 | 0.296 | 2.152 | 1.861 |

INVENTOR'S PRODUCT/PBL'S PRODUCT (DDR-FRD-F1)

| | | | | | |
|---|---|---|---|---|---|
| PBL-0.5 | 6.964 | 14.322 | 7.247 | 20.571 | 9.435 |
| PBL-1.0 | 3.629 | 2.833 | 13.397 | 8.8307 | 6.539 |
| PBL-3.0 | 15.849 | 15.775 | 3.613 | 12.181 | 11.701 |
| PBL-6.0 | 3.381 | 4.155 | 3.078 | 12.802 | 8.667 |
| PBL-12.0 | 4.025 | 4.579 | 3.559 | 7.0115 | 2.531 |
| PBL-24.0 | 2.509 | 6.617 | 3.485 | 4.011 | 2.653 |

*Number of animals (N = 5, guinea pigs) used in the study denoted as N1, N2, N3, N4 and N5
0.5, 1.0, 3.0, 6.0, 12.0 & 24.0 denote time intervals in hours.

1-25. (canceled)

26. A pharmaceutical composition for topical administration providing an enhanced localization of active ingredient comprising:
at least one active ingredient, or a salt, ester, hydrate or derivative thereof;
a gelator system consisting of a blend of surfactants;
a solvent system comprising at least one oily component;
an aqueous phase comprising one or more stabilizing agents; and
optionally other pharmaceutically acceptable excipients,
wherein the blend of surfactants act as gelators of the oily component present in the solvent system forming a three dimensional network which immobilizes the solvent system wherein the surfactant gelled oily phase can accommodate the aqueous phase without changing the lipid microenvironment and gel architecture of the composition.

27. The pharmaceutical composition, according to claim 26, wherein the active ingredient is either hydrophobic or amphiphilic in nature.

28. The pharmaceutical composition according to claim 26, wherein the active ingredient is selected from a group comprising antifungals, antibacterials, immunomodulators, steroids, analgesics, anti-inflammatory agents, keratinizing agents, antimicrobials, skin nourishing or sensitizing agents, anti-psoriatic and anti-eczema drugs, used either alone or in combination thereof.

29. The pharmaceutical composition according to claim 28, wherein the active ingredient is terbinafine, or a salt, ester, hydrate or derivative thereof.

30. The pharmaceutical composition according to claim 28, wherein the active ingredient is an immunomodulator selected from tacrolimus or cyclosporine, or a salt, ester, hydrate or derivative thereof.

31. The pharmaceutical composition according to claim 28, wherein the active ingredient is a steroid selected from testosterone or hydrocortisone or a salt, ester, hydrate or derivative thereof.

32. The pharmaceutical composition according to claim 26, 27, 28, 29, 30 or 31, wherein the gelator system consisting of a blend of surfactants comprises at least two surfactants wherein at least one is a hydrophilic surfactant having an HLB value greater than or equal to about 10, and at least one lipophilic surfactant having an HLB value less than about 10, said surfactant components being present in an amount sufficient to achieve gelation of one or more oily components present in the solvent system.

33. The pharmaceutical composition according to claim 26, 27, 28, 29, 30 or 31, wherein the gelator system consisting of a blend of surfactants comprises at least two surfactants wherein both the surfactants are non-ionic.

34. The pharmaceutical composition according to claim 32, wherein the gelator system consisting of a blend of surfactants comprises at least two surfactants wherein both the surfactants are non-ionic.

35. The pharmaceutical composition according to claim 26, 27, 28, 29, 30 or 31, wherein the gelator system is present in an amount from 5% to 50% by weight of the total weight of composition.

36. The pharmaceutical composition according to claim 26, 27, 28, 29, 30 or 31, wherein the hydrophilic surfactant is selected from a group comprising polyoxyethylene alkyl ethers; polyoxyethylene sorbitan fatty acid esters; polyoxyethylene alkyl phenols; polyethylene glycol fatty acid esters; polyglycerol fatty acid esters; polyoxyethylene glycerides; polyoxyethylene sterols; polyoxyethylene vegetable oils; polyoxyethylene hydrogenated vegetable oils; propylene glycol alginate; lecithins and hydrogenated lecithins; lysolecithin and hydrogenated lysolecithins; lysophospholipids and derivatives thereof, phospholipids and derivatives thereof; salts of fatty acids; lauryl macrogolglycerides, or mixtures thereof.

37. The pharmaceutical composition according to claim 32, wherein the hydrophilic surfactant is selected from a group comprising polyoxyethylene alkyl ethers; polyoxyethylene sorbitan fatty acid esters; polyoxyethylene alkyl phenols; polyethylene glycol fatty acid esters; polyglycerol fatty acid esters; polyoxyethylene glycerides; polyoxyethylene sterols; polyoxyethylene vegetable oils; polyoxyethylene hydrogenated vegetable oils; propylene glycol alginate; lecithins and hydrogenated lecithins; lysolecithin and hydrogenated lysolecithins; lysophospholipids and derivatives thereof; phospholipids and derivatives thereof; salts of fatty acids; lauryl macrogolglycerides, or mixtures thereof.

38. The pharmaceutical composition according to claim 26, 27, 28, 29, 30 or 31, wherein the lipophilic surfactant is selected from a group comprising fatty acids; sorbitan fatty acid esters; acetylated glycerol fatty acid esters; lower alcohol fatty acids esters; transesterification products of fatty acids, glycerides, vegetable oils, hydrogenated vegetable oils, triglycerides and polyalkylene polyols; sterols and sterol derivatives; pentaerythritol fatty acid esters and polyalkylene glycol ethers; monoglycerides and acetylated, e.g. mono-and di-acetylated monoglycerides; or mixtures thereof.

39. The pharmaceutical composition according to claim 32, wherein the lipophilic surfactant is selected from a group comprising fatty acids; sorbitan fatty acid esters; acetylated glycerol fatty acid esters; lower alcohol fatty acids esters; transesterification products of fatty acids, glycerides, vegetable oils, hydrogenated vegetable oils, triglycerides and polyalkylene polyols; sterols and sterol derivatives; pentaerythritol fatty acid esters and polyalkylene glycol ethers; monoglycerides and acetylated, e.g. mono-and di-acetylated monoglycerides; or mixtures thereof.

40. A pharmaceutical composition according to claim 26, 27, 28, 29, 30 or 31, wherein the gelator system consisting of a blend of surfactants comprise a lipophilic surfactant which is a sorbitan fatty acid ester selected from a group comprising sorbitan monolaurate, sorbitan monopalmitate, sorbitan monooleate, and sorbitan monostearate; or mixtures thereof; and a hydrophilic surfactant which is a polyoxyethylene sorbitan fatty acid ester selected from a group comprising polyoxyethylene sorbitanmonolaurate, polyoxyethylene sorbitan monopalmitate, polyoxyethylene sorbitanmonooleate, and polyoxyethylene sorbitan monostearate; or mixtures thereof.

41. A pharmaceutical composition according to claim 32, wherein the gelator system consisting of a blend of surfactants comprise a lipophilic surfactant which is a sorbitan fatty acid ester selected from a group comprising sorbitan monolaurate, sorbitan monopalmitate, sorbitan monooleate, and sorbitan monostearate; or mixtures thereof; and a hydrophilic surfactant which is a polyoxyethylene sorbitan fatty acid ester selected from a group comprising polyoxyethylene sorbitanmonolaurate, polyoxyethylene sorbitan monopalmitate, polyoxyethylene sorbitanmonooleate, and polyoxyethylene sorbitan monostearate; or mixtures thereof.

42. The pharmaceutical composition according to claim 32, wherein ratio of hydrophilic surfactant to lipophilic surfactant is 1:20 to 20:1.

43. The pharmaceutical composition according to claim 26, wherein the solvent system comprises at least one oily component, and one or more other components selected from a group comprising methanol, ethanol, isopropanol, triethyl citrate, acetyl butyl citrate or triacetin; or mixtures thereof; or other hydrophilic solvents selected from a group comprising ethylene glycol, propylene glycol, glycerol, polyethylene glycol, and polyethylene glycol esters; or mixtures thereof.

44. A pharmaceutical composition according to claim 43, wherein the at least one oily component of the solvent system is selected from a group comprising natural oils, mineral oil, mono-, di-, or tri-glyceride esters of oils selected from a group consisting of medium chain triglycerides, oleic acid, ethyl oleate, ethyl caprylate, ethyl butyrate, isopropyl myiistate, soyabean oil, canola oil or their mono-and di-glycerides, aluminium monostearate, aluminium distearate, aluminium tristearate, microcrystalline wax, petroleum wax and mixtures, used either alone or in combination thereof.

45. The pharmaceutical composition according to claims 43 or 44, wherein the at least one oily component of the solvent system is a medium chain triglyceride.

46. The pharmaceutical composition according to claim 26, wherein the aqueous phase comprises one or more of water, aliphatic or aromatic alcohols, glycols, or a mixture thereof.

47. The pharmaceutical composition according to claim 26, wherein the composition is a topical formulation and the stabilizing agent is a natural, synthetic, or semi synthetic polymer which acts as a structure former and stabilizer, wherein the stabilizing agent is selected from a group comprising chitosan, poloxamer, cellulosic polymers, gums and alginates or a mixture thereof.

48. The pharmaceutical composition according to claim 47, wherein the topical formulation range from an emulsion, cream, lotion or gel.

49. The pharmaceutical composition according to claim 47, wherein the stabilizing agent is poloxamer.

50. The pharmaceutical composition according to claim 47 or 49, wherein the stabilizer is added either in the oily phase or in aqueous phase or added as an aqueous solution up to a concentration ranging from 0.1% to 20% of the total weight of the composition.

51. A pharmaceutical composition according to claim 26, wherein the other pharmaceutically acceptable excipients are selected from the group comprising preservatives, formulation aids, antioxidants, diluents, pH adjusting agents, buffering agents, tonicity modifiers, or colorants, or a mixtures thereof.

52. A process for the preparation of a pharmaceutical composition according to claim 26, which comprises the steps of:
(i) preparing the gelator system and the solvent system followed by mixing,
(ii) incorporating the active ingredient(s) into the mixture obtained in step (i),
(iii) preparing the aqueous phase comprising the stabilizer, and
(iv) mixing the material of step (ii) with the material of step (iii) with continuous stirring to obtain the desired composition.

53. A method for the treatment of fungal, bacterial or microbial infection; inflammation; autoimmune conditions; or hormonal disorders comprising administering an effective amount of a pharmaceutical composition according to claim 26 to a subject in need thereof.
